(19) United States
(12) Patent Application Publication
(10) Pub. No.: US 2006/0134665 A1
(43) Pub. Date: Jun. 22, 2006

(54) Methods for analyzing transcripts

(75) Inventors: Thomas R. Gingeras, Encinitas, CA (US); Philipp Kapranov, Sunnyvale, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/268,907

(22) Filed: Nov. 8, 2005

Related U.S. Application Data
(63) Continuation-in-part of application No. 10/741,193, filed on Dec. 19, 2003.
(60) Provisional application No. 60/484,849, filed on Jul. 2, 2003.
(60) Provisional application No. 60/626,145, filed on Nov. 8, 2004.

(51) Int. Cl. C12Q 1/68

(52) U.S. Cl. 435/6

(57) ABSTRACT

In one aspect of the invention, high density arrays are used with 5′ and 3′ RACE (rapid amplification of cDNA ends) or RAGE (rapid amplification of Genomic DNA) in tandem to detect and characterize transcripts or genomic structures.

### RELATED APPLICATIONS

This application is a continuation-in-part of U.S. patent application Ser. No. 10/741,193, filed on Dec. 19, 2003 which claims the benefit under 35 U.S.C. § 119(e) of U.S. Provisional Application Ser. No. 60/484,849, filed on Jul. 2, 2003. In addition, this application claims the benefit under 35 U.S.C. § 119(e) of U.S. Provisional Application Ser. No. 60/626,145, filed on Nov. 8, 2004. These patent applications are incorporated herein by reference in their entirety for all purposes.

This invention was made with Government support under Contract No. N01-CO-12400 awarded by the National Cancer Institute, National Institutes of Health. The Government has certain rights in the invention.

### BACKGROUND OF THE INVENTION

This application is related to biological analysis, microarray technology and bioinformatics. Obtaining full length cDNAs has previously required the use of careful technical approaches of cDNA cloning or 5′ and 3′ RACE methodology. The products of the cDNA and RACE efforts were optimally required to be a unique molecular species (i.e. a single band on a gel or a majority of clones in a library). Therefore, there is a great need in the art for additional methods for characterizing the full length transcripts.

### SUMMARY OF THE INVENTION

In one aspect of the invention, high density arrays are used with 5′ and 3′ RACE (rapid amplification of cDNA ends) or RAGE (rapid amplification of Genomic DNA) in tandem to detect and characterize transcripts or genomic structures. The RACE can be either 3′ or 5′ RACE. The products of RACE or RAGE can be analyzed using oligonucleotide probes, preferably immobilized on a substrate to form high density oligonucleotide probe arrays. The arrays may be genomic tiling arrays, resequencing arrays and other suitable arrays.

Exemplary applications of the methods of the invention include: 1) identify the location of 5′ and 3′ end termini of transcripts (detection and characterization of alternative 5′ and 3′ termini); 2) determination of the structure of full length cDNAs; 3) detection and characterization of alternative splice isoforms for related transcripts; 4) determination of the stand or origin for transcripts; 5) the capability of pooling multiple (>2) RACE reactions to allow for gene discovery and characterization in a high throughput fashion; 6) the capability to carry out the above five tasks using low copy number transcripts by PCR amplifying the products of the RACE reactions; 7) the capability to detect associations of transcripts (exons) which are derived from transcription emanating at great distances from one another; and 8) By extension unique deletions, translocation and rearrangements can be identified by RACE like reactions using genomic DNA as the template.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 shows an exemplary analysis method of the invention.

FIG. 2 shows the structure of a well characterized gene on chromosome 22, DGSI. The gene is composed of 10 exons and is transcribed from right to left (i.e., 5′ end is on the right).

FIG. 3 shows a region of Chromosome 22 where a novel gene was characterized using RACE and array experiment.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

I. General

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W.H. Freeman Pub., New York, N.Y. and Berg et al. (2002) Biochemistry, 5th Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com. The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

II. Glossary

The following terms are intended to have the following general meanings as there used herein.

Nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about 1 M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point™ fro the specific sequence at s defined ionic strength and pH. The Tm is the temperature (under defined ionic strength, pH and nucleic acid composition) at which 50% of the probes complementary to the target sequence hybridize to the target sequence at equilibrium.

Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2nd Ed. Cold Spring Harbor Press (1989) and Anderson “Nucleic Acid Hybridization” 1st Ed., BIOS Scientific Publishers Limited (1999), which are hereby incorporated by reference in its entirety for all purposes above.

Hybridization probes are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501 filed Apr. 3, 1996.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Probe: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Effective amount refers to an amount sufficient to induce a desired result.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Single nucleotide polymorphism (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Genotyping refers to the determination of the genetic information an individual carries at one or more positions in the genome. For example, genotyping may comprise the determination of which allele or alleles an individual carries for a single SNP or the determination of which allele or alleles an individual carries for a plurality of SNPs. A genotype may be the identity of the alleles present in an individual at one or more polymorphic sites.

III. Methods for Detecting Transcript Structure

In those cases when only a portion of a full length transcript has be isolated and characterized (e.g., EST or transfrag-from Transcriptome work (www.affymetrix.com)), obtaining full length cDNAs has previously required the use of careful technical approaches of cDNA cloning or 5′ and 3′ RACE methodology. The products of the cDNA and RACE efforts were optimally required to be a unique molecular species (i.e. a single band on a gel or a majority of clones in a library). If such a unique species were not obtained it has been traditionally been seen that further analysis was unproductive.

RACE, rapid amplification of cDNA ends, is technique originally developed for analyzing full length cDNAs. 3′ RACE takes advantage of the natural poly(A) tail in mRNA as a generic priming site for PCR amplification. mRNAs are converted into cDNA using reverse transcriptase (RT) and an oligo-dT adapter primer. Specific cDNA is then amplified by PCR using a gene-specific primer (GSP) that anneals to a region of known sequences and an adapter primer that targets the poly(A) tail region.

5′ RACE, or “anchored” PCR, is a technique that facilitates the isolation and characterization of 5′ ends from low-copy messages. See, e.g., Saiki, R. K., Gelfand, D. H., Stoffel, S., Scharf, S. J., Higuchi, R., Horn, G. T., Mullis, K. B., and Erlich, H. A. (1988) Science 239, 487; Frohman, M. A., (1993) Rapid Amplification of Complementary DNA Ends for Generation of Full-Length Complementary DNAs: Thermal RACE. Methods in Enzymology 218:340-356, incorporated by reference. First strand cDNA synthesis is primed using a gene-specific antisense oligonucleotide (GSP1). This permits cDNA conversion of specific mRNA, or related families of mRNAs, and maximizes the potential for complete extension to the 5′-end of the message. Following cDNA synthesis, the first strand product is purified from unincorporated dNTPs and GSP1. TdT (Terminal deoxynucleotidyl transferase) is used to add homopolymeric tails to the 3′ ends of the cDNA. In the original protocol, tailed cDNA is then amplified by PCR using a mixture of three primers: a nested gene-specific primer (GSP2), which anneals 3′ to GSP1; and a combination of a complementary homopolymer-containing anchor primer and corresponding adapter primer which permit amplification from the homopolymeric tail. This allows amplification of unknown sequences between the GSP2 and the 5′-end of the mRNA. Similar strategy can be used to analyze genomic sequences. For example, Rapid Amplification of Genomic DNA Ends (RAGE) can be used to clone and analyze genomic sequences. See, e.g., XIANAN LIU and W. VANCE BAIRD, Rapid Amplification of Genomic DNA Ends by Nla III Partial Digestion and Polynucleotide Tailing, Plant Molecular Biology Reporter 19: 261-267, 2001 and Mizobuchi, M, Frohman, L A (1993) Rapid amplification of genomic DNA ends. Biotechniques 15: 215-216., incorporated herein by reference.

RACE may be performed using existing cDNA libraries. Random hexamer primed cDNA has also been adapted to 5′ RACE for amplification and cloning of multiple genes from a single first strand synthesis reaction. The RACE procedures may be utilized in conjunction with exon trapping methods to enable amplification and characterization of unknown coding sequences. See, e.g., Buckler, A. J., Chang, D. D., Graw, S. L., Brook, D., Haber, D. A., Sharp, P. A., and Housman, D. E. (1991) Proc. Natl. Acad. Sci USA 88, 4005, incorporated by reference.

In one aspect of the invention, transcripts are analyzed using RACE (rapid amplification of cDNA ends) and the product of RACE or samples derived from the products of RACE are hybridized with arrays designed to interrogate exons. The use of RACE and arrays allow for specific and less specific RACE products to be made (i.e. mixtures) and successfully sorted out and characterized on the arrays. This approach, also allows for high throughput analyses of multiple transcripts and genomic regions simultaneously.

RACE is a process to obtain that potentially elusive full-length cDNA. See. e.g., M. A. Frohman et al., “Rapid production of full-length cDNAs from rare transcripts: amplification using a single gene-specific oligonucleotide primer,” Proceedings of the National Academy of Sciences, 85:8998-9002, 1988. Both 5′ and 3′ RACE have been used to characterize the transcript structure. However, traditional RACE protocols are associated with high background noise due to a number of potential factors.

The use of high density arrays and 5′ and 3′ RACE (rapid amplification of cDNA ends) in tandem to detect and characterize hither to uncharacterized transcripts or genomic structures. Experiments conducted at Affymetrix shows that the quality of the RACE and subsequent RACE amplified (by PCR) reaction products can be observed to be non-specific by electrophoretic gel or cloning analyses. The arrays are able to sort though the heterogenuous mixture of amplified RACE products and display the intented region to be analyzed as a fully characterized transcript.

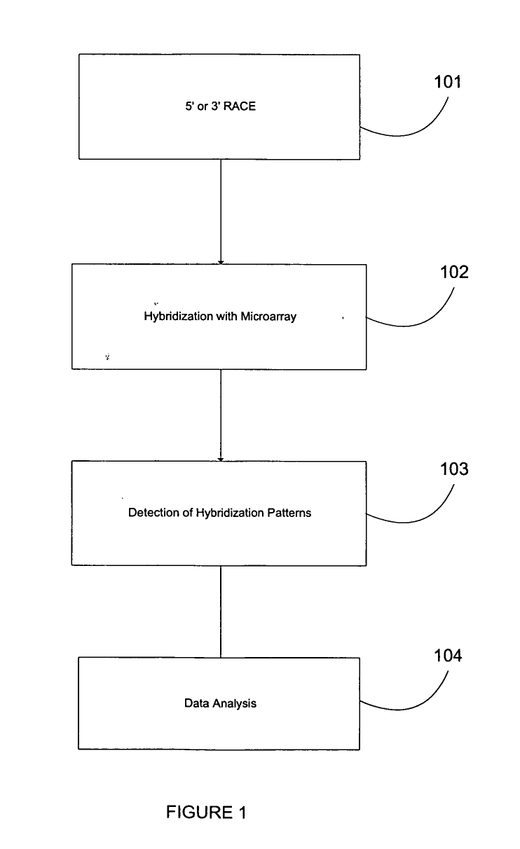

FIG. 1 shows an exemplary process for analyzing RACE products. Nucleic acid samples (such as RNAs) are used as templates for RACE reactions with suitable primers (101). Various RACE protocols are suitable including, for example, SMART methodology (Clontech), RLM-RACE Kit from Ambion and the GeneRacer™ Kit from Invitrogen.

The RACE products or nucleic acids derived from RACE products may be labeled and analyzed using high density oligonucleotide probes (102, 103), rather than traditional gel based analysis. In preferred embodiments, the probes are immobilized in arrays or in collections of encoded beads. The selection of the probes for interrogating nucleic acid sequences (such as the tiling strategy) has been described in several applications previously incorporated by references. For example, transcriptome tiling arrays are described in U.S. patent application Ser. No. ______, attorney docket number 3555.1, filed on Dec. 15, 2003, U.S. patent application Ser. No. 10/714,253, 10/712,322, all incorporated herein by reference. The transcriptome arrays typically tile the genome sequences at 1, 5, 25, 30, 35 base intervals. In some cases, the probes may be targeting transcripts from a specific strand. Resequencing arrays are also useful. Various algorithms for analyzing the hybridization data are disclosed in the applications previously incorporated by references. Custom resequencing arrays and software for analyzing the arrays are available, for example, from Affymetrix, Inc. (Santa Clara, Calif.). High throughput resequencing arrays are described in, for example, U.S. patent application Ser. Nos. 10/028,482, 10/658,879, incorporated herein by references.

The union of RACE and high density oligonucleotide array allows one to perform many analysis, includes the ability to:

1) identify the location of 5′ and 3′ end termini of transcripts (detection and characterization of alternative 5′ and 3′ termini);

2) determine the structure of full length cDNAs;

3) detect and characterize alternative splice isoforms for related transcripts;

4) determine the strand or origin for transcripts;

5) provide the capability of pooling multiple (>2) RACE reactions to allow for gene discovery and characterization in a high throughput fashion;

6) provide the capability to carry out the above five tasks using low copy number transcripts by PCR amplifying the products of the RACE reactions;

7) provide the capability to detect associations of transcripts (exons) which are derived from transcription emanating at great distances from one another; and

8) By extension unique deletions, translocation and rearrangements can be identified by RACE like reactions using genomic DNA as the template.

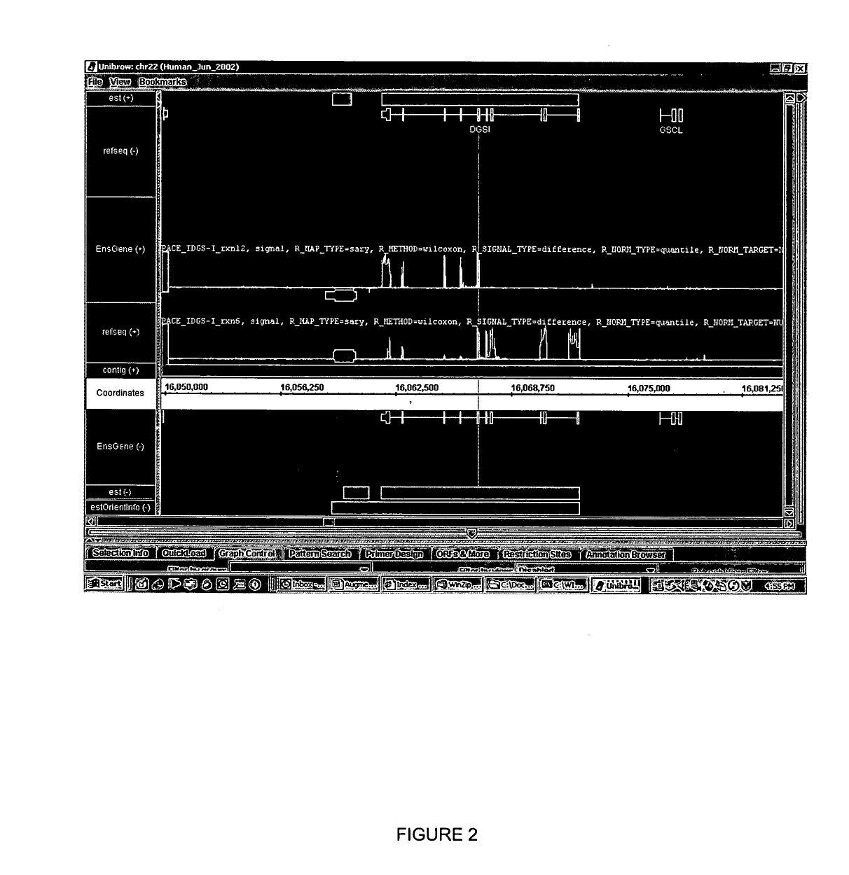

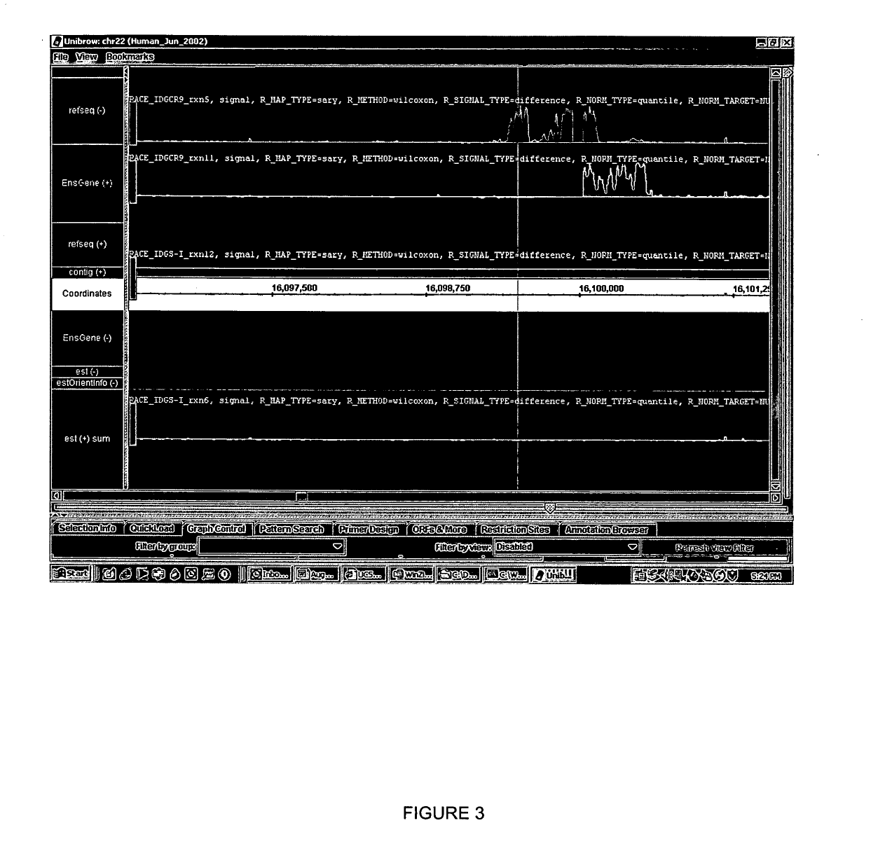

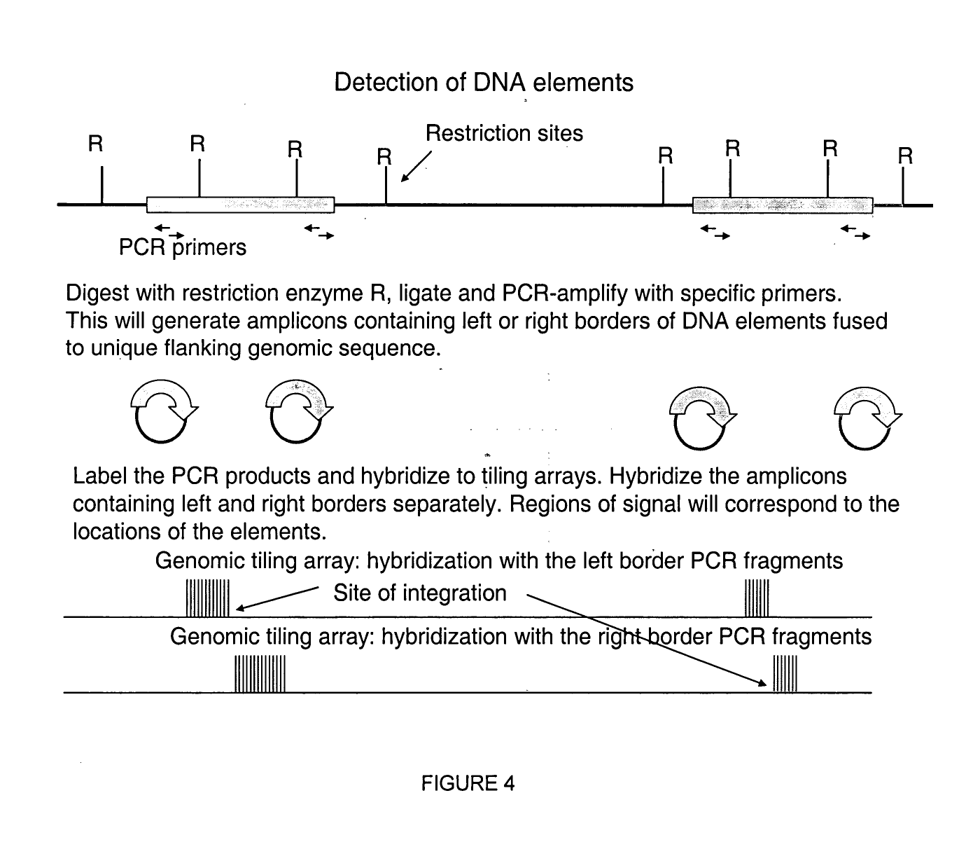

FIGS. 2 and 3 are from an example showing the use of 5′ and 3′ RACE and high density arrays to 1) confirm the structure of a previously well characterized gene and 2) characterize the full length transcript of a novel gene.

FIG. 2 is a screen capture of UniBrow visualization tool (Affymetrix, Santa Clara, Calif.) containing the structure of a well characterized gene on chromosome 22, DGSI. This gene is composed of 10 exons and is transcribed from right to left (i.e. 5′ end is on the right). Listed below are two tracks marked as DGSI-rxl2 and rx 6, respectively. There were two RACE primers in a reaction to interrogate the 5′ and 3′ ends. The primers are directed in opposite directions and positioned within exon 6 (from the 5′ end). Rx 12 track illustrates the identification of exons 6-10 and rx 6 track illustrates the identification of 1-6. Interestingly, exon 1 appears to have a longer exon than is described by the annotation for DGSI.

FIG. 3 is a screen capture containing a region of chromosome 22 which was detected as a novel gene in the transcriptome project. This was reported in Science 2002 May 3; 296: 916-919. Since then an EST (DGCR9) has been reported for this region. In the RACE and array experiment we see a positive strand transcript with 2 exons (the gap seen with DGCR9 rx 5 (upper track) is due to a lack of probes in this small region).

It is to be understood that the above description is intended to be illustrative and not restrictive. Many variations of the invention will be apparent to those of skill in the art upon reviewing the above description. All cited references, including patent and non-patent literature, are incorporated herein by reference in their entireties for all purposes.

Tiling arrays could be used to profile not only RACE, but also any RT-PCR or PCR reaction. RACE is a specific case of RT-PCR wherein gene-specific primers are positioned “outward” to determine the 5′ or 3′ sequences. However, tiling arrays could also be used to profile regular RT-PCR reactions with 2 gene-specific primers. In another embodiment of the application, as depicted in FIG. 4, a similar assay to RACE could be performed on genomic DNA to determine sites of integration of viruses, transposons or any other element that can be uniquely queried with a primer.

In some embodiments, the RACE product is first labeled and then fragmented and hybridized to the tiling arrays, we should obtain signal confined to the 5′ and 3′ ends of transcripts. In other words, using this embodiment of the protocol, the label is first incorporated only at the ends of the RACE products and in the subsequent steps the signal should be confined to the 5′ or 3′ ends of the transcripts. Therefore, this method can be used to determine 5′ and 3′ ends of transcripts.

What is claimed is:

1. A method for analyzing a nucleic acid comprising:
Performing a RACE reaction using the nucleic acid as a template; and
Analyzing the RACE reaction using a microarray.
